(19) United States
(12) Patent Application Publication
(10) Pub. No.: US 2025/0114422 A1
(43) Pub. Date: Apr. 10, 2025

(54) COMPOSITION FOR LIPOLYSIS COMPRISING EXTRACT OF MORUS ALBA L. BARK AND MAGNOLIA OFFICINALIS BARK AS ACTIVE INGREDIENT

(71) Applicant: BOINBIO-CONVERGENCE CO., LTD., Seoul (KR)

(72) Inventors: Woong Mo YANG, Hanam-si (KR); Mi Hye KIM, Seoul (KR)

(73) Assignee: BOINBIO-CONVERGENCE CO., LTD., Seoul (KR)

(21) Appl. No.: 18/836,110

(22) PCT Filed: Feb. 9, 2023
(86) PCT No.: PCT/KR2023/001872

§ 371 Date: Aug. 6, 2024

(30) Foreign Application Priority Data
Feb. 9, 2022 (KR) 10-2022-0016840

(51) Int. Cl. A61K 36/605; A61K 9/00; A61K 36/575; A61P 3/04

(52) U.S. Cl. CPC A61K 36/605; A61K 9/0019; A61K 36/575; A61P 3/04; A61K 2236/331

(57) ABSTRACT

The present invention relates to a composition for lipolysis, comprising an extract of Morus alba L. bark and Magnolia officinalis bark as an active ingredient, and it was confirmed that, when a mixed extract of Morus alba L. bark and Magnolia officinalis bark, according to the present invention, was locally administered to an animal model with fat accumulation induced by a high-fat diet, the size and weight of adipose tissue in the local area to which the extract was administered were decreased. It was also confirmed that the size of adipocytes in the adipose tissue was reduced, from which it was confirmed that the extract can be used as a composition for local lipolysis.

### TECHNICAL FIELD

The present invention relates to a composition for decomposing fat comprising Morus alba L. bark and Magnolia officinalis bark extract as active ingredients.

### BACKGROUND ART

Morus alba L. bark is a medicinal material made of Morus alba L. or a root bark of the same genus, and is known to have a diuretic effect in treating cough and asthma caused by residual heat. In addition, it is used for acute pyelonephritis and edemas due to weakness, has a hypotensive effect, and is used for treating nosebleeds and hemoptysis. Its pharmacological actions include antitussive, diuretic, hypotensive, sedative, analgesic, antipyretic, antispasmodic, and antibacterial effects. The appearance of the herb is semi-tubular or band-shaped, sometimes finely longitudinally cut, with an outer surface that is white or yellowish-brown.

Meanwhile, Magnolia officinalis, is a dried bark from the trunk or root of the magnolia tree, which belongs to the Magnoliaceae family. In spring, the bark of trees that have grown for over 20 years is peeled, dried in the shade, or briefly soaked in boiling water, then dried in the sun, steamed, and sun-dried again. It has a pungent and bitter taste, and its nature is warm. Pharmacological experiments have revealed its antibacterial effects and mild diuretic action. Additionally, it is effective for digestive disorders, vomiting, diarrhea, gastroenteritis, stomach cramps, bronchitis, and asthma.

There are approximately 20 billion adipocytes in the human body, which play a role in storing or releasing energy within the mammalian body. Within adipocytes, there are complex regulatory mechanisms for the storage and release of energy. When the supply of energy significantly exceeds demand, it is stored as triglycerides in adipocytes and then broken down into glycerol and free fatty acids for use when energy is depleted.

Modern people consume excessive amounts of energy while engaging in minimal physical activity, leading to a large accumulation of adipocytes. Particularly, due to a sedentary lifestyle, fat tends to accumulate in localized areas such as the abdomen or lower body. Therefore, there is a need to develop substances with excellent fat decomposition properties, especially for localized fat decomposition.

### DISCLOSURE

### Technical Problem

An object of the present invention is to provide a composition for decomposing fats comprising a mixed extract of Morus alba L. bark and Magnolia officinalis bark as an active ingredient.

An object of the present invention is to provide a method for decomposing fats comprising the step of administering the individual with an effective amount of a composition for decomposing fats comprising a mixed extract of Morus alba L. bark and Magnolia officinalis bark as an active ingredient.

### Technical Solution

In order to achieve the above objectives, the present invention provides a composition for decomposing fats comprising a mixed extract of Morus alba L. bark and Magnolia officinalis bark as an active ingredient.

In order to achieve the above objectives, the present invention provides a method for decomposing fats comprising administering individuals with an effective amount of a composition for decomposing fats comprising a mixed extract of Morus alba L. bark and Magnolia officinalis bark as an active ingredient.

### Advantageous Effects

When the mixed extract of Morus alba L. bark and Magnolia officinalis bark of the present invention was topically administered to an animal model with fat accumulation induced by a high-fat diet, it was observed that the size and weight of the adipose tissue in the treated local area were reduced. Additionally, it was confirmed to decrease the size of adipocytes within the adipose tissue, demonstrating its potential as a composition for localized fat decomposition and suggesting its useful application in related industries.

### DESCRIPTION OF DRAWINGS

FIG. 1 is a diagram visually confirming changes in adipose tissue in the inguinal area following local injection of the mixed extract of Morus alba L. bark and Magnolia officinalis bark of the present invention in obese mice induced by a high-fat diet.

FIG. 2 is a diagram confirming the changes in inguinal adipose tissue according to local injection of single Morus alba L. bark extract, single Magnolia officinalis bark extract, and a mixed extract of Morus alba L. bark and Magnolia officinalis bark of the present invention in obese mice induced by a high-fat diet, as a result of DXA.

(MA: Morus alba L. bark extract, MO: Magnolia officinalis bark extract, Complex: Morus alba L. bark extract+Magnolia officinalis bark extract)

FIG. 3 is a diagram quantifying the ratio of inguinal fat mass following local injections of single Morus alba L. bark extract, single Magnolia officinalis bark extract, and the combined extract of Morus alba L. bark and Magnolia officinalis bark of the present invention in high-fat diet-induced obese mice.

FIG. 4 is a diagram illustrating graphically and quantitatively the inhibition ratio of inguinal fat weight following local injections of single Morus alba L. bark extract, single Magnolia officinalis bark extract, and the combined extract of Morus alba L. bark and Magnolia officinalis bark of the present invention in high-fat diet-induced obese mice

FIG. 5 is a diagram showing the histological changes in inguinal fat tissue under an optical microscope following local injections of single Morus alba L. bark extract, single Magnolia officinalis bark extract, and the combined extract of Morus alba L. bark and Magnolia officinalis bark of the present invention in high-fat diet-induced obese mice.

FIG. 6 is a graph showing the diameter ratio of inguinal fat as a graph following local injections of single Morus alba L. bark extract, single Magnolia officinalis bark extract, and the combined extract of Morus alba L. bark and Magnolia officinalis bark of the present invention in high-fat diet-induced obese mice.

FIG. 7 is a diagram graphically and quantitatively presenting the diameter ratio of inguinal fat following local injections of single Morus alba L. bark extract, single Magnolia officinalis bark extract, and the combined extract of Morus alba L. bark and Magnolia officinalis bark of the present invention in high-fat diet-induced obese mice.

### BEST MODE OF THE INVENTION

The present invention provides a composition for lipolysis (fat degradation) comprising a mixed extract of Morus alba L. bark and Magnolia officinalis bark as an active ingredient.

According to an embodiment of the present invention, the mixed extract of Morus alba L. bark and Magnolia officinalis bark may be mixed in a weight ratio of 1:1 to be extracted.

The mixed extract of Morus alba L. bark and Magnolia officinalis bark of the present invention is a concept including all extracts, fractions and purified products obtained in each step of extraction, fractionation or purification, diluents, concentrates or dry matter thereof.

According to an embodiment of the present invention, the mixed extract may be extracted with a solvent selected from the group consisting of a lower alcohol of C1 to C4, an aqueous lower alcohol solution, distilled water and an organic solvent.

More specifically, as a suitable solvent for extracting the mixed extract of Morus alba L. bark and Magnolia officinalis bark, any pharmaceutically acceptable organic solvent may be used, and water or an organic solvent may be used, but is not limited thereto, for example, various solvents such as acetone, ether, benzene, chloroform, ethyl acetate, methylene chloride, hexane and cyclohexane, including purified water, methanol, ethanol, propanol, isopropanol, butanol, and the like, may be used alone or in combination.

As the extraction method, any one of the methods such as hot water extraction, cold precipitation extraction, reflux cooling extraction, solvent extraction, steam distillation, ultrasonic extraction, elution, compression, and the like may be selected. In addition, the extract of interest may additionally perform a conventional fractionation process, or may be purified using a conventional purification method. There is no limitation on the preparation method of the mixed extract of Morus alba L. bark and Magnolia officinalis bark of the present invention, and any known method may be used.

For example, the mixed extract of Morus alba L. bark and Magnolia officinalis bark included in the composition of the present invention may be prepared in a powder state by additional processes such as vacuum distillation and freeze drying or spray drying of the primary extract extracted by the hot water extraction or solvent extraction method. In addition, the primary extract may be further purified by various chromatography such as silica gel column chromatography, thin layer chromatography, high performance liquid chromatography, and the 20 like.

Therefore, the mixed extract of Morus alba L. bark and Magnolia officinalis bark is a concept including all extracts, fractions and purified products obtained in each step of extraction, fractionation or purification, diluents, concentrates or dry matter thereof.

According to an embodiment of the present invention, the composition may be administered topically.

According to an embodiment of the present invention, the composition may be administered in injection formulation.

The composition according to the present invention may suitably include suspending agents, solubilizing aids, stabilizers, isotonic agents, preservatives, anti-adsorption agents, surfactants, diluents, excipients, pH adjusting agents, analgesic agents, buffering agents, sulfur reducing agents, antioxidants, etc. For example, it may include sterile water, physiological saline solutions, conventional buffering agents (phosphoric acid, citric acid, other organic acids, etc.), stabilizers, salts, antioxidants (ascorbic acid, etc.), surfactants, suspending agents, isotonic agents, preservatives, etc. As an aqueous solution for injection, for example, isotonic solutions including physiological saline solutions, glucose, or other adjuvants, for example, d-sorbitol, d-mannose, d-mannitol, sodium chloride, and also buffering agents, for example, phosphate buffering agents, sodium acetate buffering agents, analgesic agents, for example, procaine hydrochloride, stabilizers, for example, benzyl alcohol, phenol, antioxidants, may be combined. Pharmaceutically acceptable carriers and preparations suitable for the present invention are described in detail in Remington's Pharmaceutical Sciences, 19th ed., 1995.

The preferable dosage of the extract contained in the composition according to the present invention varies depending on the condition and weight of the patient, the degree of disease, the form of the drug, the administration route and the period of time, but may be appropriately selected by those skilled in the art. In general, 0.001 mg to 100 mg may be administered once to humans, and preferably 0.01 mg to 10 mg may be administered once.

The term “injection” used in the present invention is a medication method that injects a medicine solution into a part of the body using a syringe and a needle to act locally or systemically, and can accurately and quickly obtain the effects of the medicine than conventional oral administration, suppository administration, or external preparation administration, and can effectively deliver the medicine to the body even when the medicine cannot be administered by the mouth or in a situation where it is difficult to deform or absorb the medicine by a digestive solution. Injection is largely intradermal injection, subcutaneous injection, intramuscular injection, intravenous injection, or intraarterial injection. Intradermal injection is a method in which absorption is slow and reactions can be seen, and it can be performed in the diagnosis or prevention of disease or can confirm side effects of antibiotics before surgery. Subcutaneous injection refers to a method of administering medicine to a subcutaneous connective tissue, and absorption is fast, and intramuscular injection is a method in which medicine is injected into muscle tissues of hips or upper arms, and medicine can be rapidly absorbed by a high blood vessel distribution of muscle. Intravenous injection refers to a method of administering medicine directly into intravenous, and a method of administering medicine can be used when medicine cannot be injected directly into muscle or subcutaneously due to the irritation of medicine.

The injectable agent for use in the above injection refers to aqueous, water-soluble, oily, suspending, emulsifying, and solid (after dissolving) formulations, and refers to a formulation that is injectable for administration so that drugs are directly acting in the body without going through the digestive tract. As a condition of the injection, it can be used as an injection if it is 1) free of excipients, 2) completely sterile, 3) free of a pyrogenic substance, 4) has an osmotic pressure similar to serum osmotic pressure, 5) a pH similar to serum pH, and 6) a formulation that does not chemically react with the components of body tissue.

In addition, the injection preparation may include solvents, dissolution aids, buffers, isotonic agents, stabilizers, sulfuric acid agents, painless agents, and suspending agents as additives according to the FDA guidelines. The additives that can be incorporated into the injection are substances other than the active ingredients contained in the formulation, which are used to increase the usefulness of pharmaceuticals, facilitate formulation, promote stabilization of the formulation, and improve their appearance. Additives may include excipients, stabilizers, preservatives, buffers, flavoring agents, suspending agents, emulsifiers, fragrances, solubilizing aids, colorants, and thickeners as necessary. However, the additive used means that it does not exhibit direct pharmacological effects at the dosage of the formulation and is safe, and does not alter the therapeutic effects of the formulation or interfere with the test, and can be summarized as follows.
1) Enhancement of stability and bioavailability.
2) Preservation or maintenance of the quality of the formulation during use.
3) Enhancing drug economy by adjusting the physical properties of the pharmaceutical.

The additives may be incorporated include distilled water for injection, 0.9% sodium chloride injection, Ringgel injection, dextrose injection, dextrose+sodium chloride injection, peg, lactide Ringgel injection, ethanol, propylene glycol, non-volatile oil-sesame oil, cottonseed oil, peanut oil, soybean oil, corn oil, oleic acid ethyl acetate, isopropyl myristate, and benzoic acid benzene. Sodium benzoate, sodium salicylate, sodium acetate, urea, urethane monoethylacetamide, butazolidine, propylene glycol, twins, nicutinamide, hexamine, dimethylacetamide, and the like may be incorporated into the solution aid. The rock buffer rock may include weak acid and salts thereof (acetic acid and sodium acetate), weak base and salts thereof (ammonia and ammonium acetate), organic compounds, proteins, albumin, peptone, and gums. Sodium chloride may be incorporated into the lake lake, and sodium bisulfite (NaHSO₃), carbon dioxide gas, sodium metabisulfite (Na₂S₂O₃), sodium sulfite (Na₂SO₃), nitrogen gas (N₂), and ethylenediaminetetraacetic acid may be incorporated into the lake stabilizer lake. Sodium bisulfide 0.1%, sodium formaldehyde, sulfoxylate, thiourea, disodium ethylenediaminetetraacetate, and acetonesodium bisulfite may be incorporated as the sulfur oxidizer. Benzyl alcohol, chlorobutanol, hydrochloric acid procaine, glucose, and calcium gluconate may be incorporated as a suspending agent, and CMC sodium, sodium alginate, Tween 80, and aluminum monostearate may be incorporated as a suspending agent.

The composition according to the present invention may be administered to a local site, and the site is not limited thereto, but may be preferably applied to the abdomen, under the jaw, the forearm, the thigh, the waist, and the hip site.

The local administration refers to administering pharmaceutical ingredients to the muscle or subdermal location of a patient by a non-systemic route, or around the local location. Therefore, local administration excludes administration through a systemic route such as intravenous or oral administration.

The unit dose of the composition according to the present invention may be from 0.1 mL to 500 mL in total with respect to the affected area, and preferably from 1 mL to 200 mL, more preferably from 1 mL to 100 mL.

The composition according to the present invention may be administered by setting several target sites (points) at regular intervals with respect to the affected area when administered once, and the total amount may refer to the total amount of dose administered through these several target sites once. The target sites may be set in the range of 1 to 50, preferably 2 to 30, more preferably 3 to 10 with respect to one affected area. In addition, the composition of the present invention includes all administered to one target site to one affected area when administered once, and it is obvious to those skilled in the art that the total amount is calculated based on the amount of the target site.

The composition of the present invention may be administered in a dose of 0.01 to 20 mL per target site (point), and preferably administered in a dose of 0.01 to 10 mL, more preferably 0.1 to 1 mL, but is not limited thereto.

The composition of the present invention may be administered once or multiple times to the target site (point), and preferably administered at intervals of from 1 week to up to 2 months.

According to an embodiment of the present invention, the composition may be administered into the subcutaneous adipose tissue.

According to an embodiment of the present invention, the composition may reduce the weight of adipocytes, and

According to an embodiment of the present invention, the size of adipocytes may be reduced.

The present invention provides a method of lipolysis, comprising administering an effective amount of a composition for lipolysis comprising a mixed extract of Morus alba L. bark and Magnolia officinalis bark as an active ingredient to a subject.

The present invention also provides a method of lipolysis, comprising administering a pharmaceutically effective amount of the mixed extract of Morus alba L. bark and Magnolia officinalis bark to a subject. The pharmaceutical composition of the present invention is administered in a therapeutically effective amount or a pharmaceutically effective amount. The term “pharmaceutically effective amount” refers to an amount sufficient to treat a disease at a reasonable benefit/risk ratio applicable to medical treatment, and the effective dose level may be determined according to the subject type and severity, age, sex, drug activity, drug sensitivity, administration time, route and excretion ratio, treatment period, factors including drugs used simultaneously, and other factors well known in the medical field.

### Modes of the Invention

Hereinafter, the present invention will be described in more detail with reference to the following Examples. However, the following Examples are only intended to embody the contents of the present invention, and the present invention is not limited thereto.

### Manufacturing Example 1. Preparation of Mixed Extracts of Morus alba L. Bark and Magnolia officinalis Bark

In order to prepare the extracts of Morus alba L. bark and Magnolia officinalis bark of the present invention, Morus alba L. bark and Magnolia officinalis bark were purchased from Dongyang Herb (Seoul, Korea). Then, 30 g of Morus alba L. bark and 30 g of Magnolia officinalis bark were placed in distilled water and extracted. After extraction, the obtained extracts were filtered using filter paper, concentrated at 60° C. using a rotary evaporator, and then freeze-dried. The obtained samples were stored at −20° C. for further experiments.

Additionally, in the high-fat diet animal model, the single Morus alba L. bark extract was administered at 0.7 mg/mL, the single Magnolia officinalis bark extract at 1.3 mg/mL, and the combined extract of Morus alba L. bark and Magnolia officinalis bark of the present invention at 2 mg/mL.

### Preparation Example 1. Local Injection of Mixed Extracts of Morus alba L. Bark and Magnolia officinalis Bark in the High Fat Diet Animal Model

The mixed extract of Morus alba L. bark and Magnolia officinalis bark of the present invention was tested for its ability to decompose fat when injected into a localized area using a high-fat diet animal model. Specifically, 5-week-old male C57BL/6J mice weighing 19-20 g were purchased and acclimated for one week with free access to food and water under a 12-hour light/dark cycle, at an indoor temperature of 22±2° C., and 50±5% humidity. To induce obesity, a high-fat diet containing 60% fat (High Fat Diet: HFD, Research Diets, D12492) was provided for 10 weeks. After 11 weeks of high-fat diet treatment, the mixed extract of Morus alba L. bark and Magnolia officinalis bark was administered three times a week for a total of 18 injections over 6 weeks. Specifically, 100 μl of the extract prepared at a concentration of 2 mg/ml was locally injected into the left inguinal adipose tissue of the mice. To assess the effect of the local injection, an equal volume of physiological saline was injected into the right inguinal adipose tissue. The right inguinal adipose tissue (Vehicle) of each mouse served as the control compared to the left inguinal adipose tissue (sample).

### Example 1. Verification of Fat Tissue Weight Reduction by Mixed Extract of Morus Alba L. Bark and Magnolia officinalis Bark

To determine whether the mixed extract of Morus alba L. bark and Magnolia officinalis bark of the present invention is more effective at reducing localized fat compared to single Morus alba L. bark extract and Magnolia officinalis bark extract, the weight of adipose tissue was measured. Specifically, mice from Preparation Example 1 were humanely sacrificed, and after dissection, the morphological changes in the left and right inguinal adipose tissues were examined. Dual-energy X-ray absorptiometry (DEXA, DXA) software was used to obtain mapping images of body composition.

Subsequently, the left and right inguinal adipose tissues were collected and weighed using an electronic scale (PAG214, OHAUS Pioneer).

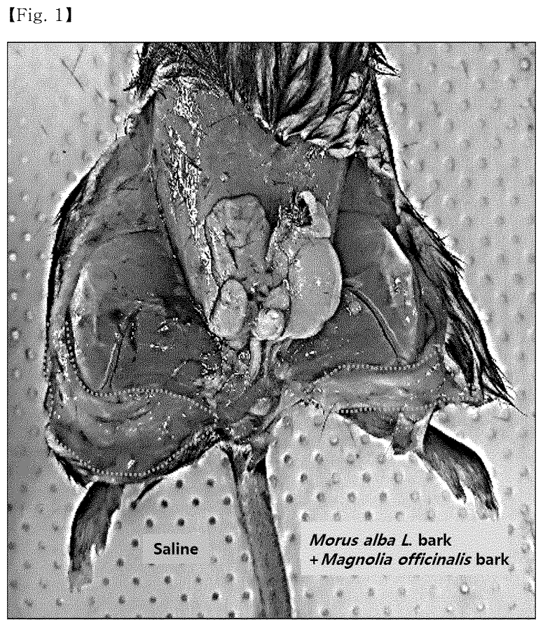

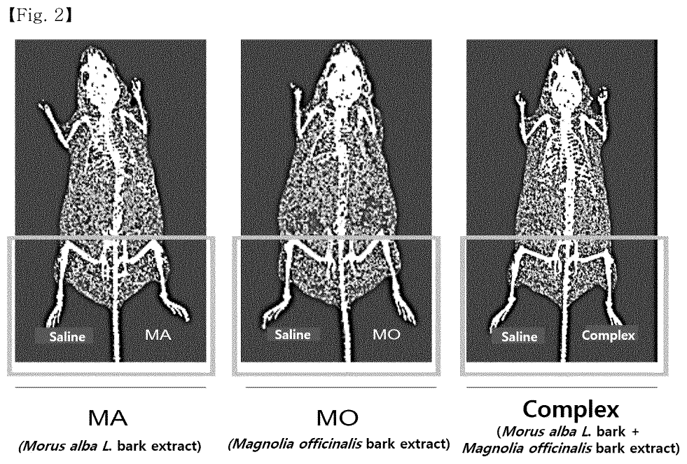

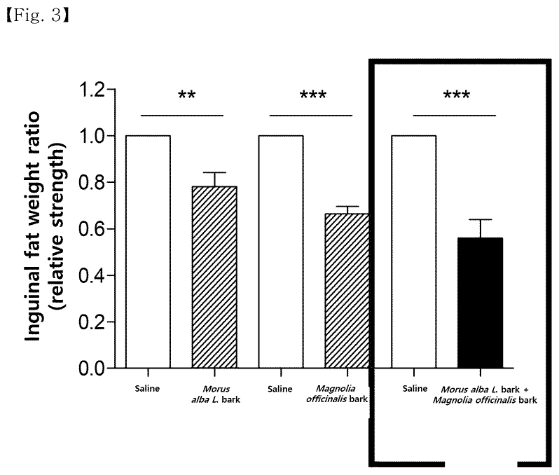

The results showed that compared to the right inguinal adipose tissue (Vehicle) of the group injected with the mixed extract of Morus alba L. bark and Magnolia officinalis bark, there was a reduction in adipose tissue in the left inguinal adipose tissue (Sample) (FIG. 1). Additionally, DXA results confirmed that the left inguinal adipose tissue in the group injected with the mixed extract of Morus alba L. bark and Magnolia officinalis bark reduced adipose tissue more effectively than those injected with single Morus alba L. bark extract or Magnolia officinalis bark extract (FIGS. 2 and 3).

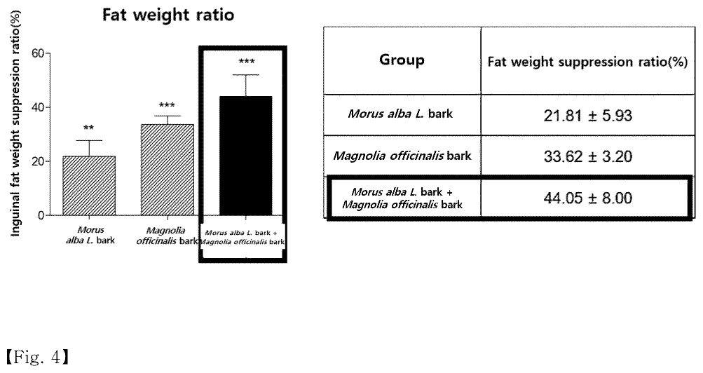

Furthermore, as shown in FIG. 4, when the weight of the right inguinal adipose tissue (Vehicle) in the groups injected with single Morus alba L. bark extract, single Magnolia officinalis bark extract, and the mixed extract of Morus alba L. bark and Magnolia officinalis bark was set to 1, the ratio of the left inguinal adipose tissue (Sample) was found to be reduced by 21.81% for the Morus alba L. bark extract, 33.62% for the Magnolia officinalis bark extract, and 44.05% for the mixed extract.

Therefore, when comparing the fat weight reduction effects between the groups injected with single Morus alba L. bark extract or Magnolia officinalis bark extract and those injected with the mixed extract of Morus alba L. bark and Magnolia officinalis bark, it suggests that the mixed extract of Morus alba L. bark and Magnolia officinalis bark of the present invention is more effective in reducing fat weight.

### Example 2. Histological Changes in Adipose Tissue by Mixed Extract of Morus alba L. Bark and Magnolia officinalis Bark

To assess the effects of the mixed extract of Morus alba L. bark and Magnolia officinalis bark on adipose tissue and cells compared to single Morus alba L. bark extract and Magnolia officinalis bark extract, histological analysis was performed on the left and right inguinal adipose tissues obtained from mice sacrificed in Example 1.

Specifically, the obtained inguinal adipose tissues were fixed in 10% paraformaldehyde for 24 hours, thoroughly washed to remove the fixative that had infiltrated the tissues, and then processed through dehydration with 70%, 90%, 95%, and 100% alcohol. The tissues were then embedded in paraffin blocks using xylene. The paraffin blocks were sectioned at 5 μm intervals to prepare slides, which were then subjected to deparaffinization and hydration processes before staining with hematoxylin & eosin (H&E) solution to observe histological changes. The diameter of adipocytes was measured using the Image J program, and the diameters of inguinal adipocytes were normalized to the control group (Vehicle) set as 1.

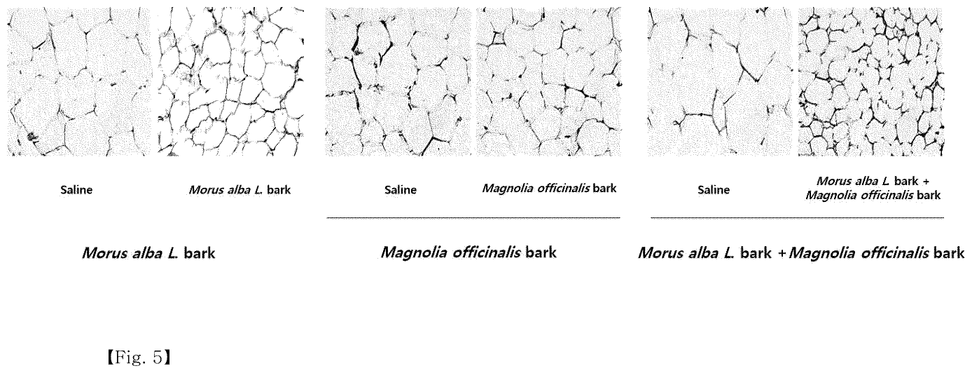

As shown in FIG. 5, the mixed extract of Morus alba L. bark and Magnolia officinalis bark resulted in a reduction in adipose tissue size in the left inguinal region (Sample) compared to the right inguinal region (Vehicle). Specifically, the mixed extract reduced adipose tissue size by 41.59% compared to 10.18% for the Morus alba L. bark extract and 34.44% for the Magnolia officinalis bark extract (FIG. 5).

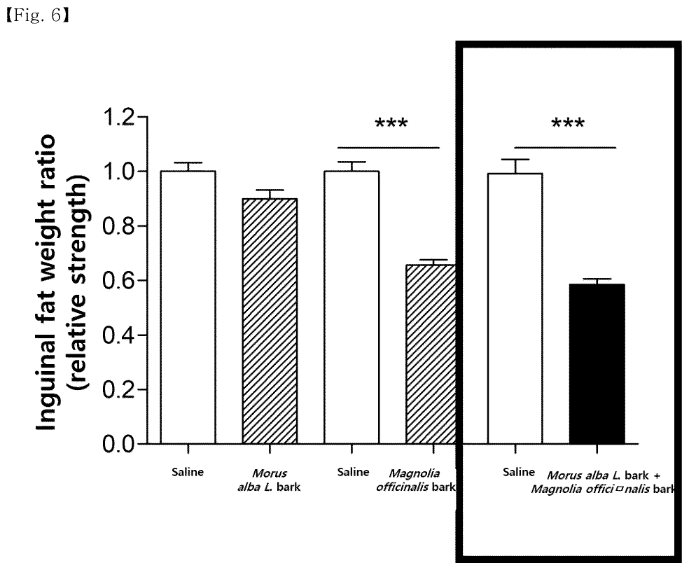

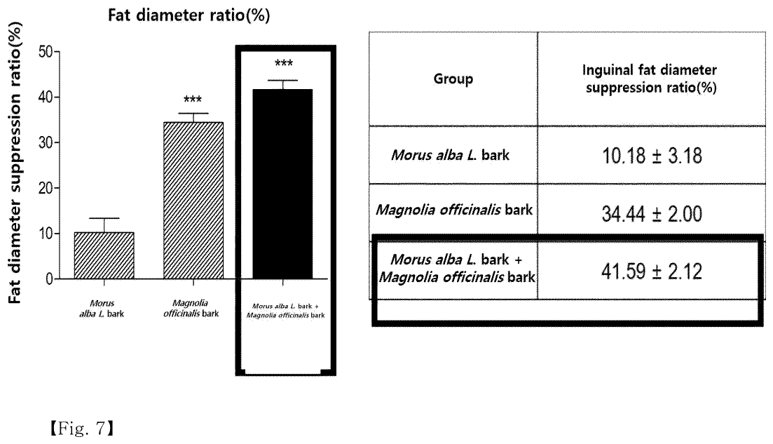

Furthermore, the mixed extract of Morus alba L. bark and Magnolia officinalis bark significantly reduced the diameter ratio of adipocytes compared to both single extracts. The diameter ratio of adipocytes in the left inguinal region was markedly reduced (FIG. 6), and the mixed extract achieved the greatest reduction in adipose tissue size compared to single extracts (FIG. 7).

Therefore, it was confirmed that the mixed extract of Morus alba L. bark and Magnolia officinalis bark of the present invention, when locally administered to a high-fat diet-induced animal model, decreases the size and weight of adipose tissue at the local injection site. Additionally, it was demonstrated to reduce the size of adipocytes within adipose tissue, establishing its efficacy as a composition for localized fat reduction.

1. A method for inducing lipolysis, the method comprising administering to a subject in need thereof a composition comprising a mixed extract of Morus alba L. bark and Magnolia officinalis bark as an active ingredient.

2. The method of claim 1, wherein the Morus alba L. bark and the Magnolia officinalis bark are mixed in a weight ratio of 1:1 to 100:100, and extracted.

3. The method of claim 1, wherein the mixed extract is extracted with a solvent selected from the group consisting of a lower alcohol of C1 to C4, an aqueous lower alcohol solution, distilled water, and an organic solvent.

4. The method of claim 1, wherein the composition is administered locally.

5. The method of claim 1, wherein the composition is administered in an injection formulation.

6. The method of claim 1, wherein the composition is administered into a subcutaneous adipose tissue.

7. The method of claim 1, wherein the subject requires reducing a weight of adipose cells.

8. The method of claim 1, wherein the subject requires reducing a size of adipose cells.

9. (canceled)
